(19) United States
(12) Patent Application Publication
(10) Pub. No.: US 2023/0173265 A1
(43) Pub. Date: Jun. 8, 2023

(54) GARMENT PROVIDING A BIASING FORCE ON A TRANSDUCER ARRAY

(71) Applicant: Novocure GmbH, Root D4 (CH)

(72) Inventors: Uri WEINBERG, Haifa (IL); Yoram WASSERMAN, Haifa (IL)

(21) Appl. No.: 18/062,372

(22) Filed: Dec. 6, 2022

Related U.S. Application Data
(60) Provisional application No. 63/288,172, filed on Dec. 10, 2021.

(51) Int. Cl. A61N 1/32; A41D 13/12; A61N 1/04

(52) U.S. Cl. CPC A61N 1/32; A41D 13/1245; A61N 1/0476

(57) ABSTRACT

A system and method are herein described. The system comprises two or more transducer arrays, each of the two or more transducer arrays comprising one or more first connector; a garment comprising a supporting layer and one or more second connector; a pump configured to selectively provide a fluid at one or more predetermined pressure; an inflatable component comprising one or more bladder and supported by the supporting layer at a location associated with the treatment area, the one or more bladder biased to a neutral position and configured to expand to an inflated position based in part on the one or more predetermined pressure, the one or more bladder in the inflated position providing a biasing pressure against a particular transducer array to cause the particular transducer array to conform to a subject's body; and a tube configured to provide fluid communication between the pump and the inflatable component.

### CROSS REFERENCE TO RELATED APPLICATIONS

This application is a non-provisional application claiming benefit to the U.S. Provisional Application No. 63/288,172 filed on Dec. 10, 2021. The entire content of the above-referenced application is hereby incorporated herein by reference in its entirety.

### BACKGROUND

Tumor Treating Fields (TTFields or TTFs) are low-intensity (e.g., 1-3 V/cm) alternating electric fields within the intermediate frequency range (e.g., 50 kHz-1 MHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the transducer arrays that make up each of these pairs are positioned on opposite sides of the body part that is being treated. More specifically, for the OPTUNE® system, one pair of electrodes of the transducer array is located to the left and right (LR) of the tumor, and the other pair of electrodes of the transducer array is located anterior and posterior (AP) to the tumor. TTFields are approved for the treatment of glioblastoma multiforme (GBM), and may be delivered, for example, via the OPTUNE° system (Novocure Limited, St. Helier, Jersey), which includes transducer arrays placed on the patient's shaved head. More recently, TTFields therapy has been approved as a combination therapy with chemotherapy for malignant pleural mesothelioma (MPM) and may find use in treating tumors in other parts of the body.

Each transducer array used for the delivery of TTFields in the OPTUNE® device comprises a set of non-conductive ceramic disk electrodes, which are coupled to the patient's skin (such as, but not limited to, the patient's shaved head for treatment of GBM). To form the ceramic disk electrodes, a conductive layer is formed on a top surface of nonconductive ceramic material. The nonconductive ceramic material is a safety feature to ensure that direct-current signals are blocked from unintentionally being transmitted to the patient by mistake. One approach to applying the TTFields in different directions is to apply the field between a first set of electrodes for a period of time, then applying a field between a second set of electrodes for a period of time, then repeating that cycle for an extended duration (e.g., over a period of days or weeks).

It is preferred that good electrical contact between the transducer arrays and the skin is maintained for the full treatment period. However, the area of the patient's body on which a particular transducer array is applied may have a surface that is concave (e.g., above a breast or below a skin fold), which may make it difficult to maintain good electrical contact for the full treatment period. Because of this difficulty in maintaining good electrical contact between the transducer array and the concave surface of the patient's body, mechanisms for maintaining good electrical contact between the transducer arrays and the patient's skin are desired. It is to garments that maintain good electrical contact between the transducer arrays and the patient's skin and methods of producing and using the same that the present disclosure is directed.

### SUMMARY OF THE DISCLOSURE

In one embodiment, the problem of maintaining good electrical contact between the transducer arrays and the concave surface of the patient's body is solved by a system comprising: two or more transducer arrays configured to deliver TTFields to a treatment area on or in a body of a subject, each of the two or more transducer arrays comprising one or more first connector; a garment comprising a supporting layer and one or more second connector disposed on a skin-facing surface of the supporting layer and configured to attach to the one or more first connector; a pump configured to selectively provide a fluid at one or more predetermined pressure; an inflatable component comprising one or more bladder and supported by the supporting layer at a location associated with the treatment area, the one or more bladder biased to a neutral position and configured to expand to an inflated position or contract to a deflated position based in part on the one or more predetermined pressure, the one or more bladder in one of the inflated position or the deflated position providing a biasing pressure against a particular transducer array of the two or more transducer arrays to cause the particular transducer array to conform to the body of the subject; and a tube configured to provide fluid communication between the pump and the inflatable component.

The details of one or more implementations of the subject matter described in this specification are set forth in the accompanying drawings and the description below. Other aspects, features, and advantages of the subject matter will become apparent from the description, the drawings, and the claims.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale, or in schematic in the interest of clarity and conciseness. Not every component may be labeled in every drawing. Like reference numerals in the figures may represent and refer to the same or similar element or function. In the drawings:

FIG. 1A is a front view of an exemplary embodiment of a system constructed in accordance with the present disclosure, the system comprising one or more inflatable component, each comprising a bladder;

FIG. 1B is a side view of the system shown in FIG. 1A;

FIG. 2A is a front view of an exemplary embodiment of the system constructed in accordance with the present disclosure, the system comprising one or more inflatable component, each comprising a plurality of bladders;

FIG. 2B is a perspective view of the system shown in FIG. 2A, showing an attachment between a garment connector and an array connector;

FIG. 3A is a cross-sectional view of the system shown in FIG. 1A, taken along the line A-A′ and in the direction of the arrows, showing a bladder in a neutral position;

FIG. 3B is a cross-sectional view of the system shown in FIG. 1A, taken along the line A-A′ and in the direction of the arrows, showing the bladder in an inflated position;

FIG. 4A is a cross-sectional view of the system shown in FIG. 2A, taken along the line B-B′ and in the direction of the arrows, showing the plurality of bladders in a neutral position;

FIG. 4B is a cross-sectional view of the system shown in FIG. 2A, taken along the line B-B′ and in the direction of the arrows, showing the plurality of bladders in an inflated position;

FIG. 5A is a cross-sectional view of a tube shown in FIG. 3A, taken along the line C-C′ and in the direction of the arrows;

FIG. 5B is a cross-sectional view of a tube shown in FIG. 4A, taken along the line D-D′ and in the direction of the arrows;

FIG. 6 is a block diagram of an exemplary embodiment of a system constructed in accordance with the present disclosure;

FIG. 7 is a process flow diagram of a method of making an exemplary embodiment of a garment constructed in accordance with the present disclosure;

FIG. 8 is a process flow diagram of a method of using an exemplary embodiment of a garment constructed in accordance with the present disclosure;

FIG. 9 is a front view of an exemplary embodiment of an inflatable component constructed in accordance with the present disclosure, showing the inflatable component adhered to a patient's skin such that a bladder is formed by the inflatable component and the patient's skin;

FIG. 10A is a cross-sectional view of the inflatable component shown in FIG. 9, taken along the line E-E′ and in the direction of the arrows, showing the bladder in a neutral position; and

FIG. 10B is a cross-sectional view of the inflatable component shown in FIG. 9, taken along the line E-E′ and in the direction of the arrows, showing the bladder in a deflated position.

### DETAILED DESCRIPTION

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise.

The use of the term “at least one” will be understood to include one as well as any quantity more than one. In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “certain embodiments,” “one example,” “for example,” or “an example” means that a particular element, feature, structure, or characteristic described in connection with the embodiment is included in at least one embodiment.

Throughout this application, the term “about” is used to indicate that a value includes the inherent variation of error for a composition/apparatus/device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Expressly included are combinations that contain repeats of one or more item or term, such as BB, AAA, AAB, BBC, AAABCCCC, CBBAAA, CABABB, and so forth.

As used herein, the term “substantially” means that the subsequently described event or circumstance completely occurs or that the subsequently described event or circumstance occurs to a great extent or degree. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the term “patient” is used interchangeably with the term “subject” and will be understood to include human and veterinary subjects.

Circuitry, as used herein, may be analog and/or digital components, or one or more suitably programmed processors (e.g., microprocessors) and associated hardware and software, or hardwired logic. Also, “components” may perform one or more functions.

As used herein, the term TTField (TTFields, or TTF(s)) refers to low-intensity (e.g., 1-4 V/cm) alternating electric fields of medium frequencies (about 50 kHz-1 MHz, and more preferably from about 100 kHz-300 kHz) that when applied to a conductive medium, such as a human body, via electrodes may be used, for example, to treat tumors as described in U.S. Pat. No. 7,016,725, 7,089,054, 7,333,852, 7,565,205, 7,805,201, and 8,244,345 by Palti and in a publication by Kirson (see Eilon D. Kirson, et al., Disruption of Cancer Cell Replication by Alternating Electric Fields, Cancer Res. 2004 64:3288-3295); all of which are hereby incorporated by reference in their entirety.

As used herein, the term TT Signal is an electrical signal that, when received by electrodes applied to a conductive medium, such as a human body, causes the electrodes to generate the TTField described above. The TT Signal is often an AC electrical signal.

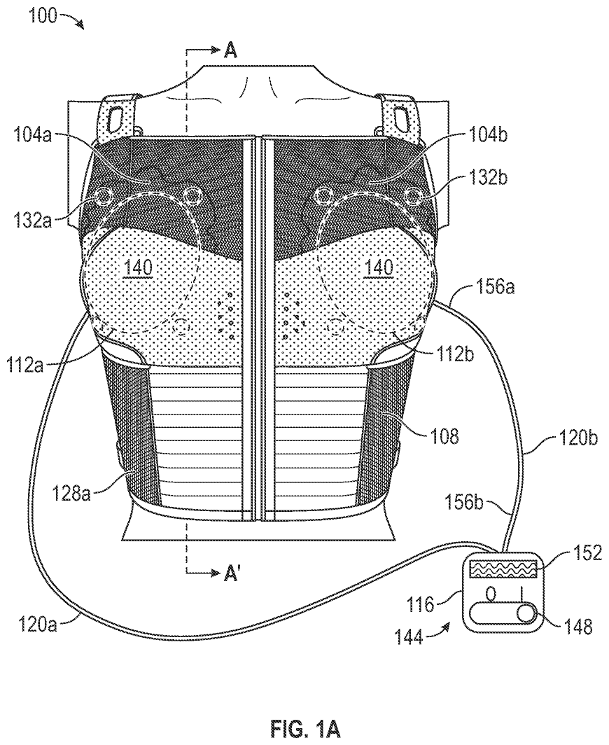

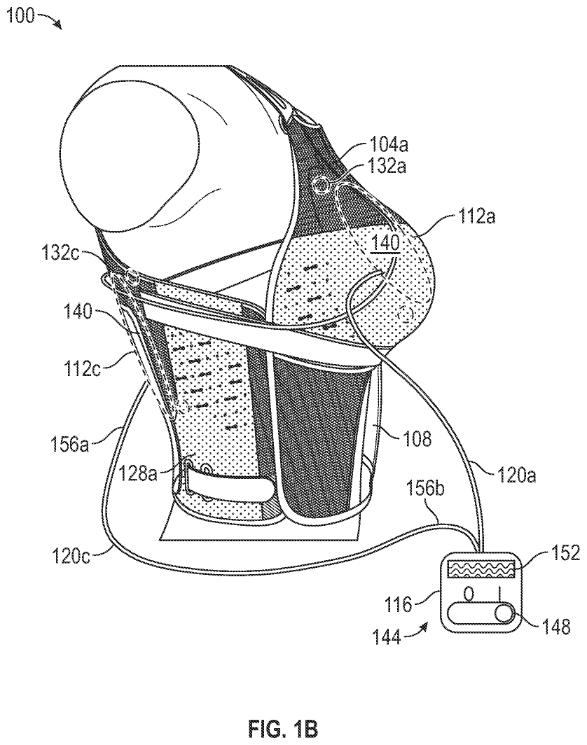

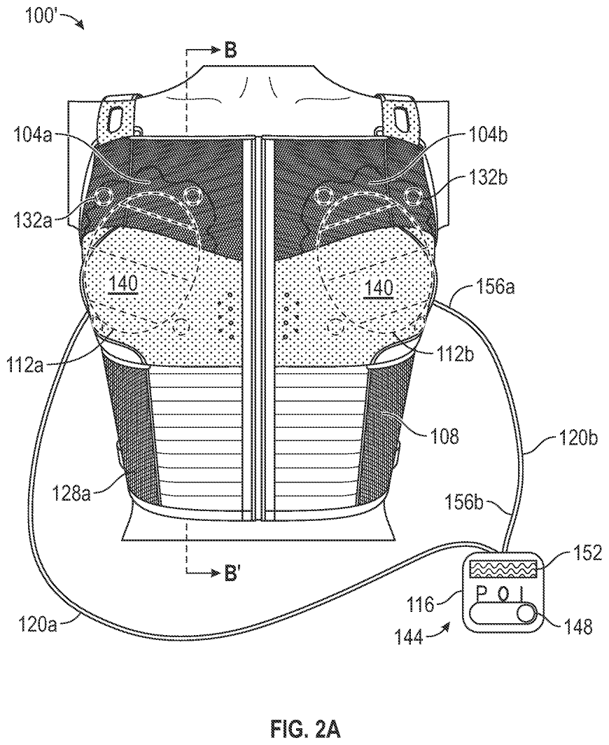

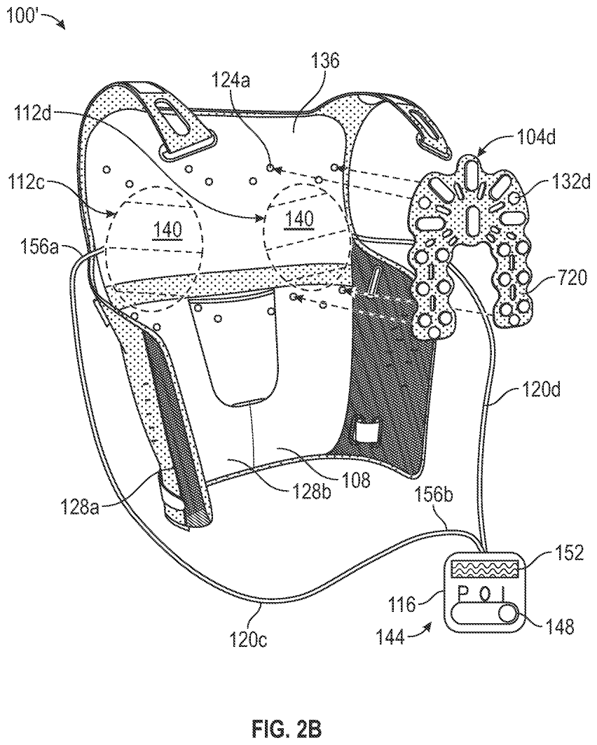

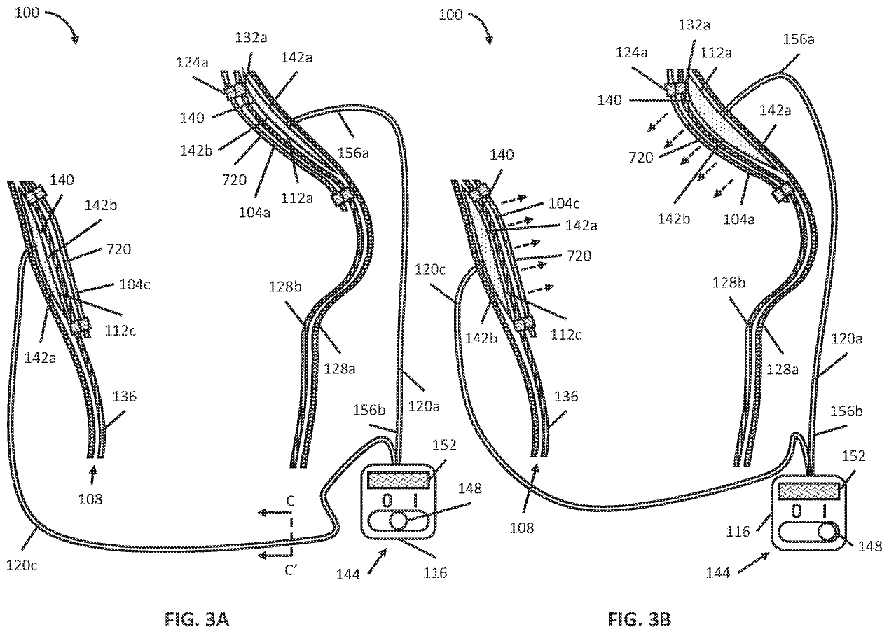

Referring now to the drawings and in particular to FIG. 1A and FIG. 1B, shown therein is an exemplary embodiment of a system 100 comprising two or more transducer arrays 104a-n (which may include, for example, a first transducer array 104a, a second transducer array 104b, a third transducer array 104c (see FIG. 3A), a fourth transducer array (not shown), etc.), a garment 108, one or more inflatable component 112a-n (which may include, for example, a first inflatable component 112a, a second inflatable component 112b, a third inflatable component 112c, a fourth inflatable component (not shown), etc.), a pump 116, and one or more tube 120a-n (which may include, for example, a first tube 120a, a second tube 120b, a third tube 120c, a fourth tube (FIG. 2B), etc.).

The two or more transducer arrays 104a-n may be configured to deliver TTFields to a treatment area on or in a body of a subject. At least one of the two or more transducer arrays 104a-n may comprise one or more array connector 132a-n. For purposes of clarity, only one of the one or more array connector 132a-n of each of the two or more transducer arrays 104a-n is labeled with the reference numeral 132a-n. The one or more array connector 132a-n may be disposed on and connected to a garment-facing surface of each of the two or more transducer arrays 104a-n. The two or more transducer arrays 104a-n may be disposed at two or more locations associated with the treatment area.

The two or more transducer arrays 104a-n may generate an alternating electric field within the treatment area. The treatment area typically encompasses at least one tumor, and the generation of the alternating electric field selectively destroys or inhibits growth of the tumor. The two or more transducer arrays 104a-n may be externally applied to a patient; that is, the two or more transducer arrays 104a-n may be applied to the patient's skin in an area devoid of a wound so that the two or more transducer arrays 104a-n do not cover such a wound in order to apply an electric field (i.e., TTField) thereby generating current within the patient's tissue. Externally applied TTFields may be of a local type or widely distributed type; for example, one of the two or more transducer arrays 104a-n may cover a skin tumor or lesion to assist in the treatment of the skin tumor or lesion close to the surface of the patient's skin.

The garment 108 may comprise a first layer 128a, a supporting layer 128b (see FIG. 2B), and one or more garment connector 124a-n (shown as garment connector 124a in FIG. 2B) connected to the first layer 128a or the supporting layer 128b. The one or more inflatable component 112a-n may be disposed between the first layer 128a and the supporting layer 128b and/or attached to the supporting layer 128b at one or more location associated with the treatment area. In the embodiment shown in FIG. 1A and FIG. 1B, the garment 108 is a vest; however, in other embodiments, the garment 108 may be any type of clothing, including but not limited to a shirt, a jacket, a coat, a cap, a hat, a helmet, a sleeve, a glove, pants, shorts, a shoe, a sandal, a boot, a sock, stockings, an undergarment, or the like. In one embodiment, the garment 108 may be, for example, constructed of a woven material (e.g., a woven fabric), a mesh material, or the like. In other embodiments, the garment 108 may be constructed of a non-woven material (e.g., a non-woven fabric). In one embodiment, the supporting layer 128b is a vest; however, the supporting layer 128b may be any type of clothing, including but not limited to a shirt, a jacket, a coat, a cap, a hat, a helmet, a sleeve, a glove, pants, shorts, a shoe, a sandal, a boot, a sock, stockings, an undergarment, or the like. In one embodiment, the supporting layer 128a may be, for example, constructed of a woven material (e.g., a woven fabric), a mesh material, or the like. In other embodiments, the supporting layer 128a may be constructed of a non-woven material (e.g., a non-woven fabric).

Each of the one or more inflatable component 112a-n may comprise a bladder 140. The bladder 140 may be constructed of a flexible, fluid-impermeable material, such as rubber, silicone, polymer, or the like. The bladder 140 may comprise at least two side walls (i.e., a first side wall 142a and a second side wall 142b) (see FIG. 3A) surrounding an inner cavity.

In certain embodiments, the bladder 140 may be biased to a neutral position. In certain embodiments, the one or more inflatable component 112a-n may comprise a biasing material (e.g., a sponge) positioned within the bladder 140 such that the bladder 140 in the neutral position may provide an initial biasing pressure against a particular transducer array 104 of the two or more transducer arrays 104a-n to press the particular transducer array 104 onto the patient's skin.

The pump 116 may be configured to selectively provide a fluid at one or more predetermined pressure to the bladder 140 of each of the one or more inflatable component 112a-n. The fluid may be a liquid (such as, for example, a saline solution), a gas (such as, for example, air), or any other fluid substance. In the embodiment shown in FIG. 1A, the pump 116 is part of an electronic apparatus 144 comprising a controller 148 and a display 152.

While the controller 148 shown in FIG. 1A is a switch, the controller 148 in other embodiments may be, for example, a dial, a keypad, or the like. In the embodiment shown in FIG. 1A, the controller 148 is configured to be movable between an “off” position and an “on” position; however, in other embodiments, the controller 148 may be configured to be movable to other positions, such as, for example, a “reverse” position or a “pulse” position, both of which are described in more detail below.

The controller 148 in the “on” position may send signals to a processor 712 (see FIG. 6) of the electronic apparatus 144, which may cause the processor 712 to send signals to the pump 116 to cause the pump 116 to provide fluid to the bladder 140 of each of the one or more inflatable component 112a-n at one or more predetermined pressure in order to expand the bladder 140 to the inflated position. The controller 148 in the “off” position may send signals to the processor 712, which may cause the processor 712 to cease sending signals to the pump 116, thereby causing the pump 116 to cease providing fluid to the bladder 140.

In certain embodiments, the controller 148 in the “on” position (or, in particular embodiments, the “reverse” position) may send signals to the processor 712, which may cause the processor 712 to send signals to the pump 116 to cause the pump 116 to provide fluid to—or draw fluid from—the bladder 140 of each of the one or more inflatable component 112a-n in order to modify the pressure within the bladder 140 such that, while the controller 148 remains in the “on” position (or, in particular embodiments, the “reverse” position), the pressure within the bladder 140 is maintained in a steady state.

The display 152 may be configured to provide notifications to the user, which may include, for example, an alert, an alarm, or the like. The display 152 may be configured to provide information to the user, including but not limited to the current position of the controller 148 (e.g., the “on” position, the “off” position, the “reverse” position, or the “pulse” position), the particular predetermined pressure at which fluid is being provided by the pump 116, an amount of battery life remaining for the electronic apparatus 144, a time remaining until the electronic apparatus 144 has a full battery (if charging) or an empty battery (if in use), a progression of the treatment, a description of a particular notification, or the like. The user may be a medical professional, such as a doctor, nurse, therapist, or other person acting under the instruction of a doctor, nurse, or therapist, or the patient.

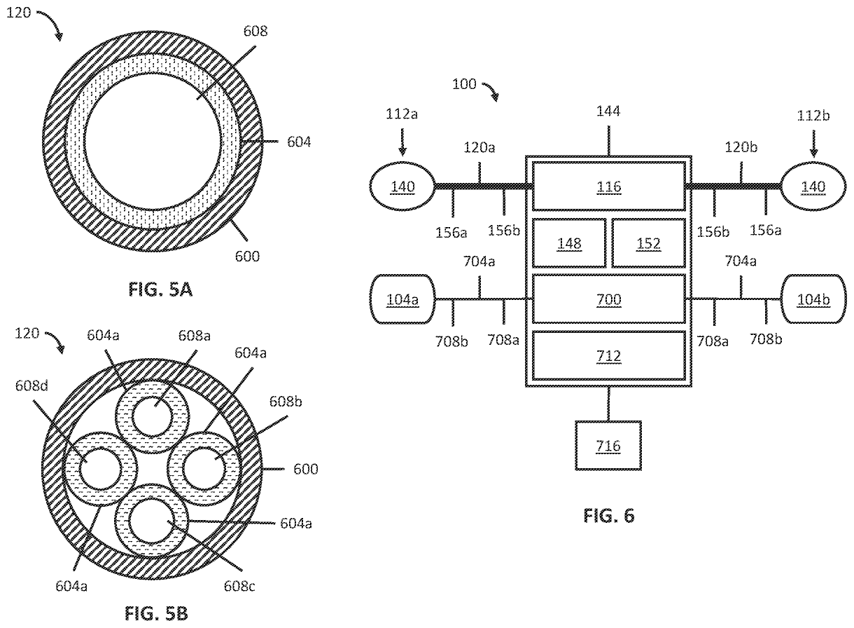

As is described in more detail below, each of the one or more tube 120a-n may comprise an outer covering 600 (see FIG. 5A) and a hose 604 (see FIG. 5A) disposed within the outer covering 600, the hose 604 surrounding a fluid pathway 608 (see FIG. 5A) configured to provide fluid communication between the pump 116 and the bladder 140 of each of the one or more inflatable component 112a-n.

Each of the one or more tube 120a-n may comprise a first end 156a and a second end 156b. For purposes of clarity, only one of the one or more tube 120a-n is labeled with the reference numerals 156a and 156b. The first end 156a of each of the one or more tube 120a-n may be coupled to the bladder 140 of each of the one or more inflatable component 112a-n. The second end 156b of each of the one or more tube 120a-n may be coupled to the pump 116 of the electronic apparatus 144.

Referring now to FIG. 2A and FIG. 2B, shown therein is another exemplary embodiment of the system 100′ wherein each of the one or more inflatable component 112a-n comprise a plurality of bladders 140a-n. For purposes of clarity, only one of the plurality of bladders 140a-n of each of the one or more inflatable component 112a-n is labeled with the reference numeral 140.

As is described in more detail below, each of the one or more tube 120a-n may comprise an outer covering 600 (see FIG. 5B) and a plurality of hoses 604a-n (see FIG. 5B) disposed within the outer covering 600, the plurality of hoses 604a-n surrounding a plurality of fluid pathways 608a-n (see FIG. 5B) configured to provide fluid communication between the pump 116 and a particular bladder 140 of the plurality of bladders 140a-n.

As shown in FIG. 2B, the one or more garment connector 124a-n may be disposed on a skin-facing surface 136 of the supporting layer 128b at one or more location adjacent to the two or more locations associated with the treatment area. The one or more garment connector 124a-n may be configured to attach to the one or more array connector 132a-n.

In the embodiment shown in FIG. 2A and FIG. 2B, the controller 148 is configured to be movable between an “off” position, an “on” position, and a “pulse” position; however, in other embodiments, the controller 148 may be configured to be movable to other positions, such as, for example, a “reverse” position, which is described in more detail below.

The controller 148 in the “on” position may send signals to the processor 712 of the electronic apparatus 144, which may cause the processor 712 to send signals to the pump 116 to cause the pump 116 to provide fluid to each of the plurality of bladders 140a-n of each of the one or more inflatable component 112a-n at one or more predetermined pressure in order to expand each of the plurality of bladders 140a-n to the inflated position.

In certain embodiments, the controller 148 in the “on” position (or, in particular embodiments, the “reverse” position) may send signals to the processor 712, which may cause the processor 712 to send signals to the pump 116 to cause the pump 116 to provide fluid to—or draw fluid from—each of the plurality of bladders 140a-n of each of the one or more inflatable component 112a-n in order to modify the pressure within the plurality of bladders 140a-n such that, while the controller 148 remains in the “on” position (or, in particular embodiments, the “reverse” position), the pressure within the plurality of bladders 140a-n is maintained in a steady state.

The controller 148 in the “off” position may send signals to the processor 712, which may cause the processor 712 to cease sending signals to the pump 116, thereby causing the pump 116 to cease providing fluid to or drawing fluid from each of the plurality of bladders 140a-n.

The controller 148 in the “pulse” position may send signals to the processor 712, which may cause the processor 712 to send signals to the pump 116 to cause the pump 116 to provide fluid to—and draw fluid from—each of the plurality of bladders 140a-n (via each of the plurality of fluid pathways 608a-n) in order to modify the pressure within each of the plurality of bladders 140a-n such that, while the controller 148 remains in the “pulse” position, the pressure within at least one of the plurality of bladders 140a-n is variable (e.g., oscillating between a high-pressure state and a low-pressure state, beginning in a low-pressure state and increasing to a high-pressure state, beginning in a high-pressure state and decreasing to a low-pressure state, etc.).

FIG. 2B shows a manner in which the one or more garment connector 124a-n and the one or more array connector 132a-n may be used to attach a particular transducer array 104 of the two or more transducer arrays 104a-n to the garment 108. In the embodiment shown in FIG. 2B, the one or more garment connector 124a-n and the one or more array connector 132a-n are snap fasteners; however, in other embodiments, the one or more garment connector 124a-n and the one or more array connector 132a-n may be any attachment mechanism, including but not limited to snaps, buttons, ties, zippers, hook-and-loop material, flaps, double-sided tape, or the like.

Referring now to FIG. 3A and FIG. 3B, shown therein is a cross-sectional view of the system 100 shown in FIG. 1A. taken along the line A-A′ and in the direction of the arrows. FIG. 3A shows the controller 148 in the “off” position and the bladder 140 of each of the one or more inflatable component 112a-n in the neutral position. FIG. 3B shows the controller 148 in the “on” position and the bladder 140 of each of the one or more inflatable component 112a-n in the inflated position. As shown in FIG. 3B, the bladder 140 may be configured to expand to the inflated position based in part on the one or more predetermined pressure at which the fluid is provided by the pump 116 when the controller 148 is in the “on” position. The bladder 140 in the inflated position may provide a biasing pressure against a particular transducer array 104 of the two or more transducer arrays 104a-n to cause the particular transducer array 104 to conform to the body of the subject.

The two or more transducer arrays 104a-n may be flexible and configured to conform to the body of the subject in response to pressure being applied to the two or more transducer arrays 104a-n via the bladder 140 in the inflated position (or, in certain embodiments, the deflated position).

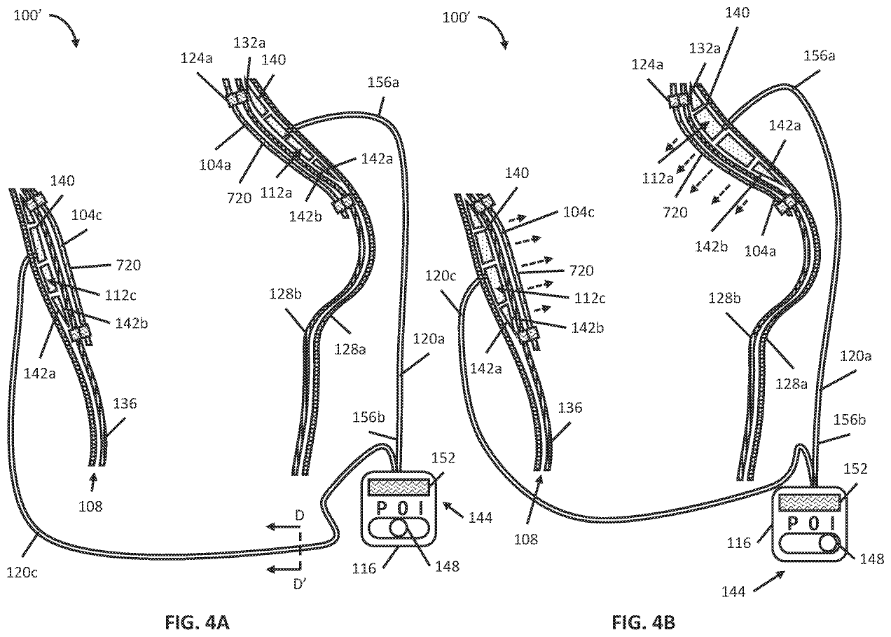

Referring now to FIG. 4A and FIG. 4B, shown therein is a cross-sectional view of the system 100′ shown in FIG. 2A. taken along the line B-B′ and in the direction of the arrows. FIG. 4A shows the controller 148 of the electronic apparatus 144 in the “off” position and the plurality of bladders 140a-n of each of the one or more inflatable component 112a-n in the neutral position. For purposes of clarity, only one of the first side wall 142a and only one of the second side wall 142b are labeled with the reference numerals 142a and 142b. FIG. 4B shows the controller 148 in the “on” position and the plurality of bladders 140a-n of each of the one or more inflatable component 112a-n in the inflated position.

As shown in FIG. 4B, the plurality of bladders 140a-n may be configured to expand to the inflated position based in part on the one or more predetermined pressure at which the fluid is provided by the pump 116 when the controller 148 is in the “on” position. The plurality of bladders 140a-n in the inflated position may provide a biasing pressure against a particular transducer array 104 of the two or more transducer arrays 104a-n to cause the particular transducer array 104 to conform to the body of the subject.

Shown in FIG. 5A is a cross-sectional view of a particular tube 120 of the one or more tube 120a-n shown in FIG. 3A, taken along the line C-C′ and in the direction of the arrows. As shown in FIG. 5A, in certain embodiments, each of the one or more tube 120a-n may comprise an outer covering 600 and a hose 604 disposed within the outer covering 600. The hose 604 may surround a fluid pathway 608 configured to provide fluid communication between the pump 116 and the bladder 140 of the one or more inflatable component 112a-n. The hose 604 may be constructed of a flexible, fluid-impermeable material, such as rubber, silicone, polymer, or the like.

Shown in FIG. 5B is a cross-sectional view of a particular tube 120 of the one or more tube 120a-n shown in FIG. 4A, taken along the line D-D′ and in the direction of the arrows. As shown in FIG. 5B, in certain embodiments, each of the one or more tube 120a-n may comprise the outer covering 600 and a plurality of hoses 604a-n disposed within the outer covering 600, the plurality of hoses 604a-n surrounding a plurality of fluid pathways 608a-n. Thus, when four hoses 604a-n are positioned within the outer covering 600, as is shown in FIG. 5B, four fluid pathways 608a-n are provided (which may include, for example, a first fluid pathway 608a, a second fluid pathway 608b, a third fluid pathway 608c, a fourth fluid pathway 608d, etc.). Each of the plurality of fluid pathways 608a-n may be configured to provide fluid communication between the pump 116 and a particular bladder 140 of the plurality of bladders 140a-n of each of the one or more inflatable component 112a-n.

Referring now to FIG. 6, shown therein is a block diagram of the system 100. As shown in FIG. 6, the system 100 may comprise the electronic apparatus 144 configured to actuate the pump 116 to provide fluid at one or more predetermined pressure to each of the one or more bladder 140a-n of each of the one or more inflatable component 112a-n. As discussed above, the electronic apparatus 144 may include the controller 148 and the display 152.

In the embodiment shown in FIG. 6, the electronic apparatus 144 further includes an electric field generator 700 configured to generate electrical signals (i.e., TT signals) in the shape of waveforms or trains of pulses as an output. The electric field generator 700 may be configured to generate an alternating voltage waveform (i.e., TTField) in the range of about 50 kHz to about 1 MHz (preferably from about 100 kHz to about 300 kHz). The required voltages are such that an electric field intensity in the patient's tissue within the treatment area is in the range of about 0.1 V/cm to about 10 V/cm. To achieve this electric field, the potential difference between, for example, the first transducer array 104a and the second transducer array 104b, is determined by the relative impedances of the patient's body and the connection between the two or more transducer arrays 104a-n and the patient's body.

The two or more transducer arrays 104a-n may be supplied with the electrical signals (i.e., TT signals). The two or more transducer arrays 104a-n, being supplied with the electrical signals, may cause an electrical current to flow between the transducer array 104a and the transducer array 104b. The electrical current may cause an electric field (i.e., TTField) having a frequency and an amplitude to be generated between the transducer array 104a and the transducer array 104b. In such an embodiment, the electronic apparatus 144 may control the output of the electric field generator 700 causing the output to remain constant at a value predetermined by the user or set by the manufacturer.

The two or more transducer arrays 104a-n may be coupled to the electric field generator 700 via two or more conductive leads 704a-n (which may include, for example, a first conductive lead 704a, a second conductive lead 704b, etc.). The two or more conductive leads 704a-n may be standard isolated conductors with a flexible metal shield, and are preferably grounded, thereby preventing spread of any electrical field generated by the two or more conductive leads 704a-n. Each of the two or more conductive leads 704a-n may comprise a first end 708a and a second end 708b. The first end 708a of each of the two or more conductive leads 704a-n may be configured to connect to the electric field generator 700. The second end 708b of each of the two or more conductive leads 704a-n may be configured to connect to a particular transducer array 104 of the two or more transducer arrays 104a-n.

In certain embodiments, to protect the patient from any current due to DC voltage or DC offset voltage passing through the patient, each of the two or more conductive leads 704a-n may include a DC blocking component (such as, for example, a blocking capacitor) to block DC current from passing to the two or more transducer arrays 104a-n.

Optionally, the electronic apparatus 144 may include a processor 712 and an impedance sensor 716 coupled to the electronic apparatus 144, the impedance sensor 716 configured to monitor an impedance between the two or more transducer arrays 104a-n and the patient's skin. If, while the system 100 is in use, the impedance between the two or more transducer arrays 104a-n and the patient's skin rises above a predetermined threshold (as measured by the impedance sensor 716), the impedance sensor 716 may send signals to the processor 712, which may cause the processor 712 to send signals to the pump 116 to cause the pump 116 to provide additional fluid to a particular bladder 140 of the one or more bladder 140a-n at one or more elevated pressure in order to further expand the particular bladder 140 to a hyperinflated position, for example. The particular bladder 140 in the hyperinflated position may provide a stronger biasing pressure against a particular transducer array 104 of the two or more transducer arrays 104a-n, which may have the effect of improving electrical contact between the particular transducer array 104 and the patient's skin. In certain embodiments, once the particular bladder 140 is further expanded and while the controller 148 remains in the “on” position (or, in certain embodiments, the “reverse” position), the pressure within the particular bladder 140 may be maintained in a steady state; however, in other embodiments, the processor 712 may send signals to the pump 116 to cause the pump 116 to draw fluid from the particular bladder 140 in order to gradually release the pressure unless and until the impedance between the two or more transducer arrays 104a-n and the patient's skin drops below the predetermined threshold (as measured by the impedance sensor 716).

In certain embodiments, each of the two or more transducer arrays 104a-n may include a skin-facing surface 720 devoid of any adhesives (such as, for example, a tape, a gel, a glue, a paste, etc.) which would otherwise hold the two or more transducer arrays 104a-n in place against the patient's skin. In such embodiments, the two or more transducer arrays 104a-n may be held in place against the patient's skin by the garment 108. In certain embodiments, one or more of the two or more transducer arrays 104a-n and the garment 108 may be packaged within a container (e.g., box, blister pack, or the like) and sold as part of a kit.

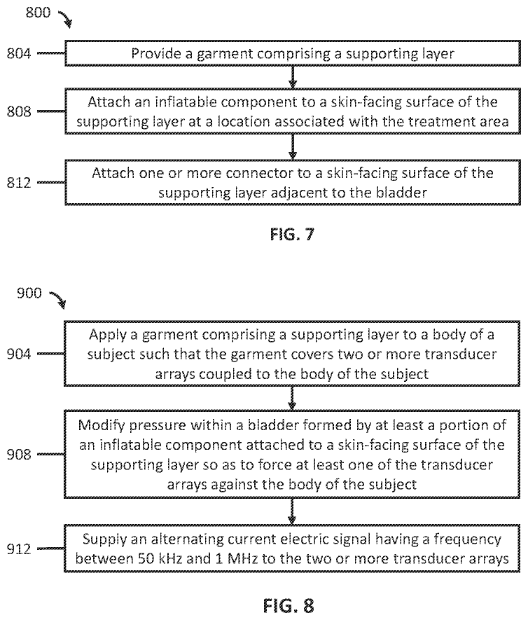

Referring now to FIG. 7, shown therein is a process flow diagram of a method 800 of making the garment 108 as disclosed herein. The method 800 may comprise the steps of:

providing a garment 108 comprising a supporting layer 128b (step 804); attaching an inflatable component 112 to a skin-facing surface 136 of the supporting layer 128b at a location associated with the treatment area (step 808); and attaching one or more garment connector 124a-n to a skin-facing surface 136 of the supporting layer 128b adjacent to the bladder 140 (step 812).

The method may further comprise the step of attaching two or more transducer arrays 104a-n to the supporting layer 128b of the garment 108, each of the two or more transducer arrays 104a-n comprising one or more array connector 132a-n configured to attach to the one or more garment connector 124a-n, the two or more transducer arrays 104a-n configured to deliver TTFields to the treatment area.

The method may further comprise the step of coupling the inflatable component 112 to a pump 116 configured to provide a fluid at one or more predetermined pressure. The step of coupling the inflatable component 112 to the pump 116 may further comprise coupling the inflatable component 112 to a tube 120 comprising one or more fluid pathways 608a-n and coupling the tube 120 to the pump 116. Each of the one or more fluid pathways 608a-n may be configured to provide fluid communication between the pump 116 and a particular bladder 140 of the one or more bladder 140a-n.

Referring now to FIG. 8, shown therein is a process flow diagram of a method 900 of using the garment 108 as disclosed herein. The method 900 may comprise the steps of: applying a garment 108 comprising a supporting layer 128b to a body of a subject such that the garment 108 covers two or more transducer arrays 104a-n coupled to the body of the subject (step 904); modifying pressure within a bladder 140 formed by at least a portion of an inflatable component 112 attached to a skin-facing surface 136 of the supporting layer 128b so as to force at least one of the transducer arrays 104a-n against the body of the subject (step 908); and supplying an alternating current electric signal having a frequency between 50 kHz and 1 MHz to the two or more transducer arrays 104a-n (step 912). The method 900 of using the garment 108 may be performed by the subject themselves, or by a third-party, such as a medical professional or another person, for example.

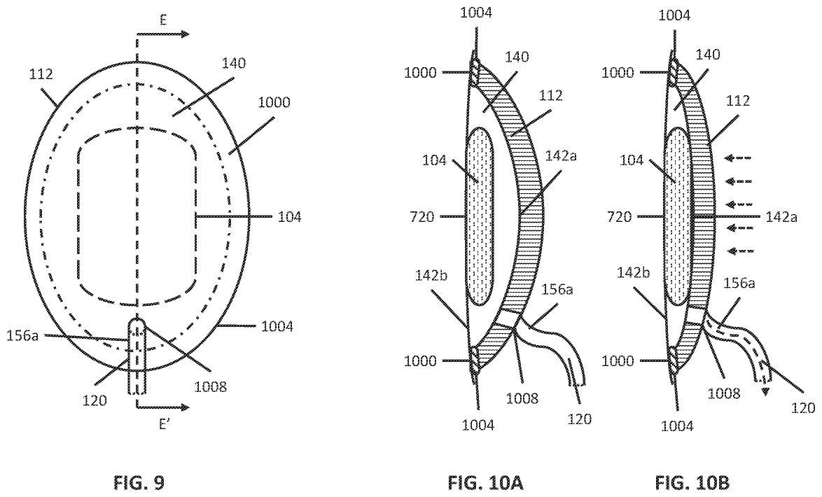

Referring now to FIG. 9, in certain embodiments, each of the one or more inflatable component 112a-n may be configured to be adhered to a material adhered to the patient's skin by an adhesive 1000, the adhesive 1000 may be disposed on an outer edge 1004 of a particular inflatable component 112 such that, when the particular inflatable component 112 is applied, an inner cavity is formed between the particular inflatable component 112 and the patient's skin, thereby forming the bladder 140 comprising the first side wall 142a (i.e., the particular inflatable component 112) (see FIG. 10A) and the second side wall 142b (i.e., the material adhered to the patient's skin) (see FIG. 10A). The bladder 140 may surround a particular transducer array 104 of the two or more transducer arrays 104a-n coupled to the patient's skin at a location associated with a treatment area. Each of the one or more inflatable component 112a-n in such embodiments may be provided with a channel 1008 to allow fluid to pass in and out of the bladder 140. A first end 156a of a particular tube 120a-n of the one or more tube 120a-n may be coupled to the channel 1008.

Referring now to FIG. 10A and FIG. 10B, shown therein is a cross-sectional view of the particular inflatable component 112 of FIG. 9, taken along the line E-E′ and in the direction of the arrows. FIG. 10A shows the bladder 140 in the neutral position, while FIG. 10B shows the bladder 140 in the deflated position. As discussed above, the controller 148 in the “reverse” position may send signals to the processor 712, which may cause the processor 712 to send signals to the pump 116 to cause the pump 116 to draw fluid from the bladder 140 of each of the one or more inflatable component 112a-n at one or more predetermined pressure in order to contract the bladder 140 to the deflated position. The bladder 140 in the deflated position may provide a suctioning force which may pull the supporting layer 128b of the garment 108 against a particular transducer array 104 of the two or more transducer arrays 104a-n to cause the particular transducer array 104 to conform to the body of the subject.

NON-LIMITING ILLUSTRATIVE EMBODIMENTS OF THE INVENTIVE CONCEPT(S)

The following is a number list of non-limiting illustrative embodiments of the inventive concepts disclosed herein:

Illustrative Embodiment 1. A system, comprising: two or more transducer arrays configured to deliver TTFields to a treatment area on or in a body of a subject, each of the two or more transducer arrays comprising one or more first connector; a garment comprising a supporting layer and one or more second connector disposed on a skin-facing surface of the supporting layer and configured to attach to the one or more first connector; a pump configured to selectively provide a fluid at one or more predetermined pressure; an inflatable component comprising one or more bladder and supported by the supporting layer at a location associated with the treatment area, the one or more bladder biased to a neutral position and configured to expand to an inflated position or contract to a deflated position based in part on the one or more predetermined pressure, the one or more bladder in one of the inflated position or deflated position providing a biasing pressure against a particular transducer array of the two or more transducer arrays to cause the particular transducer array to conform to the body of the subject;

and a tube configured to provide fluid communication between the pump and the inflatable component.

Illustrative Embodiment 2. The system of Illustrative Embodiment 1, wherein the tube comprises one or more hose surrounding one or more fluid pathway, each of the one or more fluid pathway configured to provide fluid communication between the pump and a particular bladder of the one or more bladder.

Illustrative Embodiment 3. The system of Illustrative Embodiment 1, wherein the inflatable component is a first inflatable component, the one or more bladder is one or more first bladder, the location is a first location, the particular transducer array is a first particular transducer array, and the tube is a first tube, the system further comprising: a second inflatable component comprising one or more second bladder and supported by the supporting layer at a second location associated with the treatment area, the one or more second bladder biased to a neutral position and configured to expand to an inflated position or contract to a deflated position based in part on the one or more predetermined pressure, the one or more second bladder in the inflated position or deflated position providing a biasing pressure against a second particular transducer array of the two or more transducer arrays to cause the second particular transducer array to conform to the body of the subject; and a second tube configured to provide fluid communication between the pump and the second inflatable component.

Illustrative Embodiment 4. The system of Illustrative Embodiment 3, wherein the first tube comprises one or more first hose surrounding one or more first fluid pathway, each of the one or more first fluid pathway configured to provide fluid communication between the pump and a first particular bladder of the one or more first bladder, and the second tube comprises one or more second hose surrounding one or more second fluid pathway, each of the one or more second fluid pathway configured to provide fluid communication between the pump and a second particular bladder of the one or more second bladder.

Illustrative Embodiment 5. The system of Illustrative Embodiment 1, wherein each of the one or more bladder comprises a biasing material such that the one or more bladder in the neutral position provides an initial biasing pressure against a particular transducer array of the two or more transducer arrays.

Illustrative Embodiment 6. The system of Illustrative Embodiment 1, wherein the pump is part of an electronic apparatus, the electronic apparatus having circuitry to cause the pump to provide the fluid at one or more elevated pressure during a period of time in which a measured impedance is above a predetermined threshold.

Illustrative Embodiment 7. A garment for delivering TTFields to a treatment area on or in a body of a subject, comprising: a supporting layer; one or more connector disposed on a skin-facing surface of the supporting layer adjacent to a treatment area; an inflatable component comprising one or more bladder and supported by the supporting layer at a location associated with the treatment area, the one or more bladder biased to a neutral position and configured to expand to an inflated position or contract to a deflated position based in part on one or more predetermined pressure.

Illustrative Embodiment 8. The garment of Illustrative Embodiment 7, wherein the one or more connector is configured to attach a transducer array to a skin-facing surface of the supporting layer.

Illustrative Embodiment 9. The garment of Illustrative Embodiment 7, wherein the one or more connector is one or more first connector, and further comprising two or more transducer arrays configured to deliver TTFields to the treatment area, each of the two or more transducer arrays comprising one or more second connector, the one or more second connector configured to attach to the one or more first connector.

Illustrative Embodiment 10. The garment of Illustrative Embodiment 7, wherein the inflatable component is a first inflatable component, the one or more bladder is one or more first bladder, the location is a first location, the garment further comprising: a second inflatable component comprising one or more second bladder supported by the supporting layer at a second location associated with the treatment area, the one or more second bladder biased to a neutral position and configured to expand to an inflated position or contract to a deflated position based in part on the one or more predetermined pressure.

Illustrative Embodiment 11. The garment of Illustrative Embodiment 7, further comprising a pump configured to selectively provide a fluid at one or more predetermined pressure, and a tube configured to provide fluid communication between the pump and the inflatable component.

Illustrative Embodiment 12. The garment of Illustrative Embodiment 11, wherein the tube comprises one or more hose surrounding one or more fluid pathway, each of the one or more fluid pathway configured to provide fluid communication between the pump and a particular bladder of the one or more bladder.

Illustrative Embodiment 13. The garment of Illustrative Embodiment 11, wherein the one or more bladder is one or more first bladder, the tube is a first tube, and further comprising one or more second bladder, the first tube configured to provide fluid communication between the pump and the one or more first bladder, and further comprising a second tube configured to provide fluid communication between the pump and the one or more second bladder.

Illustrative Embodiment 14. The garment of Illustrative Embodiment 9, wherein each of the one or more bladder comprises a biasing material such that the one or more bladder in the neutral position provides an initial biasing pressure against a particular transducer array of the two or more transducer arrays.

Illustrative Embodiment 15. The garment of Illustrative Embodiment 11, wherein the pump is part of an electronic apparatus, the electronic apparatus having circuitry to cause the pump to provide the fluid at one or more elevated pressure during a period of time in which a measured impedance is above a predetermined threshold.

Illustrative Embodiment 16. A method of making a garment for delivering TTFields to a treatment area on or in a body of a subject, comprising: providing a garment comprising a supporting layer; attaching an inflatable component to a skin-facing surface of the supporting layer at a location associated with the treatment area, the inflatable component comprising one or more bladder biased to a neutral position; and attaching one or more connector to a skin-facing surface of the supporting layer adjacent to the bladder.

Illustrative Embodiment 17. The method of Illustrative Embodiment 16, wherein the one or more connector is one or more first connector, and further comprising the step of attaching two or more transducer arrays to the supporting layer of the garment, each of the two or more transducer arrays comprising one or more second connector configured to attach to the one or more first connector, the two or more transducer arrays configured to deliver TTFields to the treatment area.

Illustrative Embodiment 18. The method of Illustrative Embodiment 16, further comprising the step of coupling the inflatable component to a pump configured to provide a fluid at one or more predetermined pressure, the one or more bladder configured to expand to an inflated position based in part on the one or more predetermined pressure.

Illustrative Embodiment 19. The method of Illustrative Embodiment 18, wherein the one or more bladder is further configured to contract to a deflated position based in part on the one or more predetermined pressure.

Illustrative Embodiment 20. The method of Illustrative Embodiment 18, wherein the step of coupling the inflatable component to the pump further comprises: coupling the inflatable component to a tube comprising one or more hose surrounding one or more fluid pathway, each of the one or more fluid pathway configured to provide fluid communication between the pump and a particular bladder of the one or more bladder; and coupling the tube to the pump.

Illustrative Embodiment 21. A method of delivering TTFields to a treatment area on or in a body of a subject, comprising: applying a garment comprising a supporting layer to the body of the subject such that the garment covers two or more transducer arrays coupled to the body of the subject; modifying pressure within a bladder formed by at least a portion of an inflatable component attached to a skin-facing surface of the supporting layer so as to force at least one of the transducer arrays against the body of the subject; and supplying an alternating current electric signal having a frequency between 50 kHz and 1 MHz to the two or more transducer arrays.

Illustrative Embodiment 22. The method of Illustrative Embodiment 21, further comprising the step of applying the two or more transducer arrays to the body of the subject.

Illustrative Embodiment 23. The method of Illustrative Embodiment 22, further comprising the step of coupling the two or more transducer arrays to an electric field generator before or after applying the two or more transducer arrays to the body of the subject, the electric field generator configured to generate the alternating current electric signal having the frequency between 50 kHz and 1 MHz.

Illustrative Embodiment 24. A kit for applying transducer arrays to a body of a subject, comprising: two or more transducer arrays for delivering TTFields to a treatment area on or in the body of the subject, each of the two or more transducer arrays comprising a garment-facing surface and a skin-facing surface, each transducer array of the two or more transducer arrays comprising one or more first connector connected to the garment-facing surface of the transducer array; and a garment comprising one or more second connector for attachment to the one or more first connector, the one or more second connector disposed on a skin-facing surface of the garment; wherein the skin-facing surface of each transducer array of the two or more transducer arrays is devoid of a bonding material configured to bond the transducer array to the body of the subject.

Illustrative Embodiment 25. The kit of Illustrative Embodiment 24, wherein the garment is a vest.

Illustrative Embodiment 26. A system, comprising: two or more transducer arrays configured to deliver TTFields to a treatment area on or in a body of a subject; a garment comprising a supporting layer; a pump configured to selectively provide a fluid at one or more predetermined pressure; an inflatable component attached to a skin-facing surface of the supporting layer at a location associated with the treatment area, the inflatable component comprising a bonding material disposed on an outer edge of the inflatable component such that a bladder may be formed by the inflatable component and a material adhered to the body of the subject, the bladder configured to contract to a deflated position based in part on the one or more predetermined pressure to pull the bladder against a particular transducer array of the two or more transducer arrays to cause the particular transducer array to conform to the body of the subject; and a tube configured to provide fluid communication between the pump and the inflatable component.

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

Even though particular combinations of features and steps are recited in the claims and/or disclosed in the specification, these combinations are not intended to limit the disclosure. In fact, many of these features and steps may be combined in ways not specifically recited in the claims and/or disclosed in the specification. Although each dependent claim listed below may directly depend on only one other claim, the disclosure includes each dependent claim in combination with every other claim in the claim set.

What is claimed is:

1. A system, comprising:
two or more transducer arrays configured to deliver TTFields to a treatment area on or in a body of a subject, each of the two or more transducer arrays comprising one or more first connector;
a garment comprising a supporting layer and one or more second connector disposed on a skin-facing surface of the supporting layer and configured to attach to the one or more first connector;
a pump configured to selectively provide a fluid at one or more predetermined pressure;
an inflatable component comprising one or more bladder and supported by the supporting layer at a location associated with the treatment area, the one or more bladder biased to a neutral position and configured to expand to an inflated position or contract to a deflated position based in part on the one or more predetermined pressure, the one or more bladder in one of the inflated position or deflated position providing a biasing pressure against a particular transducer array of the two or more transducer arrays to cause the particular transducer array to conform to the body of the subject; and
a tube configured to provide fluid communication between the pump and the inflatable component.

2. The system of claim 1, wherein the tube comprises one or more hose surrounding one or more fluid pathway, each of the one or more fluid pathway configured to provide fluid communication between the pump and a particular bladder of the one or more bladder.

3. The system of claim 1, wherein the inflatable component is a first inflatable component, the one or more bladder is one or more first bladder, the location is a first location, the particular transducer array is a first particular transducer array, and the tube is a first tube, the system further comprising:
a second inflatable component comprising one or more second bladder and supported by the supporting layer at a second location associated with the treatment area, the one or more second bladder biased to a neutral position and configured to expand to an inflated position or contract to a deflated position based in part on the one or more predetermined pressure, the one or more second bladder in the inflated position or deflated position providing a biasing pressure against a second particular transducer array of the two or more transducer arrays to cause the second particular transducer array to conform to the body of the subject; and
a second tube configured to provide fluid communication between the pump and the second inflatable component.

4. The system of claim 3, wherein the first tube comprises one or more first hose surrounding one or more first fluid pathway, each of the one or more first fluid pathway configured to provide fluid communication between the pump and a first particular bladder of the one or more first bladder, and the second tube comprises one or more second hose surrounding one or more second fluid pathway, each of the one or more second fluid pathway configured to provide fluid communication between the pump and a second particular bladder of the one or more second bladder.

5. The system of claim 1, wherein each of the one or more bladder comprises a biasing material such that the one or more bladder in the neutral position provides an initial biasing pressure against a particular transducer array of the two or more transducer arrays.

6. The system of claim 1, wherein the pump is part of an electronic apparatus, the electronic apparatus having circuitry to cause the pump to provide the fluid at one or more elevated pressure during a period of time in which a measured impedance is above a predetermined threshold.

7. A garment for delivering TTFields to a treatment area on or in a body of a subject, comprising:
a supporting layer;
one or more connector disposed on a skin-facing surface of the supporting layer adjacent to a treatment area; and
an inflatable component comprising one or more bladder and supported by the supporting layer at a location associated with the treatment area, the one or more bladder biased to a neutral position and configured to expand to an inflated position or contract to a deflated position based in part on one or more predetermined pressure.

8. The garment of claim 7, wherein the one or more connector is configured to attach a transducer array to a skin-facing surface of the supporting layer.

9. The garment of claim 7, wherein the one or more connector is one or more first connector, and further comprising two or more transducer arrays configured to deliver TTFields to the treatment area, each of the two or more transducer arrays comprising one or more second connector, the one or more second connector configured to attach to the one or more first connector.

10. The garment of claim 7, wherein the inflatable component is a first inflatable component, the one or more bladder is one or more first bladder, the location is a first location, the garment further comprising:
a second inflatable component comprising one or more second bladder supported by the supporting layer at a second location associated with the treatment area, the one or more second bladder biased to a neutral position and configured to expand to an inflated position or contract to a deflated position based in part on the one or more predetermined pressure.

11. The garment of claim 7, further comprising a pump configured to selectively provide a fluid at one or more predetermined pressure, and a tube configured to provide fluid communication between the pump and the inflatable component.

12. The garment of claim 11, wherein the tube comprises one or more hose surrounding one or more fluid pathway, each of the one or more fluid pathway configured to provide fluid communication between the pump and a particular bladder of the one or more bladder.

13. The garment of claim 11, wherein the one or more bladder is one or more first bladder, the tube is a first tube, and further comprising one or more second bladder, the first tube configured to provide fluid communication between the pump and the one or more first bladder, and further comprising a second tube configured to provide fluid communication between the pump and the one or more second bladder.

14. The garment of claim 9, wherein each of the one or more bladder comprises a biasing material such that the one or more bladder in the neutral position provides an initial biasing pressure against a particular transducer array of the two or more transducer arrays.

15. The garment of claim 11, wherein the pump is part of an electronic apparatus, the electronic apparatus having circuitry to cause the pump to provide the fluid at one or more elevated pressure during a period of time in which a measured impedance is above a predetermined threshold.

16. A method of making a garment for delivering TTFields to a treatment area on or in a body of a subject, comprising:
providing a garment comprising a supporting layer;
attaching an inflatable component to a skin-facing surface of the supporting layer at a location associated with the treatment area, the inflatable component comprising one or more bladder biased to a neutral position; and
attaching one or more connector to a skin-facing surface of the supporting layer adjacent to the bladder.

17. The method of claim 16, wherein the one or more connector is one or more first connector, and further comprising the step of attaching two or more transducer arrays to the supporting layer of the garment, each of the two or more transducer arrays comprising one or more second connector configured to attach to the one or more first connector, the two or more transducer arrays configured to deliver TTFields to the treatment area.

18. The method of claim 16, further comprising the step of coupling the inflatable component to a pump configured to provide a fluid at one or more predetermined pressure, the one or more bladder configured to expand to an inflated position based in part on the one or more predetermined pressure.

19. The method of claim 18, wherein the one or more bladder is further configured to contract to a deflated position based in part on the one or more predetermined pressure.

20. The method of claim 18, wherein the step of coupling the inflatable component to the pump further comprises:
coupling the inflatable component to a tube comprising one or more hose surrounding one or more fluid pathway, each of the one or more fluid pathway configured to provide fluid communication between the pump and a particular bladder of the one or more bladder; and
coupling the tube to the pump.

21. A method of delivering TTFields to a treatment area on or in a body of a subject, comprising:
applying a garment comprising a supporting layer to the body of the subject such that the garment covers two or more transducer arrays coupled to the body of the subject;
modifying pressure within a bladder formed by at least a portion of an inflatable component attached to a skin-facing surface of the supporting layer so as to force at least one of the transducer arrays against the body of the subject; and
supplying an alternating current electric signal having a frequency between 50 kHz and 1 MHz to the two or more transducer arrays.

22. The method of claim 21, further comprising the step of applying the two or more transducer arrays to the body of the subject.

23. The method of claim 22, further comprising the step of coupling the two or more transducer arrays to an electric field generator before or after applying the two or more transducer arrays to the body of the subject, the electric field generator configured to generate the alternating current electric signal having the frequency between 50 kHz and 1 MHz.

24. A kit for applying transducer arrays to a body of a subject, comprising:
two or more transducer arrays for delivering TTFields to a treatment area on or in the body of the subject, each of the two or more transducer arrays comprising a garment-facing surface and a skin-facing surface, each transducer array of the two or more transducer arrays comprising one or more first connector connected to the garment-facing surface of the transducer array; and
a garment comprising one or more second connector for attachment to the one or more first connector, the one or more second connector disposed on a skin-facing surface of the garment;
wherein the skin-facing surface of each transducer array of the two or more transducer arrays is devoid of a bonding material configured to bond the transducer array to the body of the subject.

25. The kit of claim 24, wherein the garment is a vest.

26. A system, comprising:
two or more transducer arrays configured to deliver TTFields to a treatment area on or in a body of a subject;
a garment comprising a supporting layer;
a pump configured to selectively provide a fluid at one or more predetermined pressure;
an inflatable component attached to a skin-facing surface of the supporting layer at a location associated with the treatment area, the inflatable component comprising a bonding material disposed on an outer edge of the inflatable component such that a bladder may be formed by the inflatable component and a material adhered to the body of the subject, the bladder configured to contract to a deflated position based in part on the one or more predetermined pressure to pull the bladder against a particular transducer array of the two or more transducer arrays to cause the particular transducer array to conform to the body of the subject; and
a tube configured to provide fluid communication between the pump and the inflatable component.
